(19) United States
(12) Patent Application Publication
(10) Pub. No.: US 2008/0251072 A1
(43) Pub. Date: Oct. 16, 2008

(54) Inhaler Device

(76) Inventors: Amar Lulla, Maharashtra (IN); Geena Malhotra, Maharashtra (IN)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 11/988,749

(22) PCT Filed: Jul. 13, 2006
(86) PCT No.: PCT/GB2006/002601

§ 371 Date: May 29, 2008

(30) Foreign Application Priority Data
Jul. 13, 2005 (IN) 841/MUM/2005
Jun. 15, 2006 (IN) 951/MUM/2006

(51) Int. Cl. A61M 15/00

(52) U.S. Cl. 128/203.15

(57) ABSTRACT

An inhaler device (2) is for the inhalation of particulate substances is provided. The device comprises a housing (5) having first and second openings, a first air passageway (11) being defined through said housing between said first and second openings, an extraction member (40) extending from said housing and having a second air passageway (46) formed therein, and an airflow directing means (16) proximate one of said first and second openings.

The present application relates to an inhaler device. More particularly, the present application relates to an inhaler device for administering a substance to a user. More particularly still, the present application relates to an inhaler device for administering a dry powder medicament to the lungs of a human in need thereof.

Various types of dry powder inhaler devices are known, including single dose, multiple unit dose and multi-dose devices.

Single dose inhalers generally use a capsule containing the medicament to be inserted into a receptacle area of the inhaler. The capsule has to be opened or perforated in order to allow part of the inspiratory air stream to enter the capsule for medicament entrainment or to discharge the powder from the capsule during inhalation. Generally this is achieved by opening or perforating the capsule with pins or cutting blades, to enable air to enter the capsule through a first perforation, and to enable the air entrained with medicament to exit through a second perforation. After inhalation, the emptied capsule is removed from the inhaler, such that a further capsule of medicament can be loaded for subsequent inhalation. Therefore, It is necessary to disassemble the inhaler for insertion and removal of the medicament capsules, which is an operation which can be difficult and burdensome for some patients.

In order for such a dry powder inhaler device to be successful, it is important that as much medicament as possible reaches the lungs of the user. Typically, when a powdered substance is inhaled through a mouthpiece or nozzle of a dry power inhaler, a substantial part of the active pharmaceutical agent held in the capsule fails to reach the lungs of the user. In particular, a significant quantity may be deposited in the oral cavity of the user. This can lead to a significant loss of active pharmaceutical agent during inhalation.

Problems also exist with the airborne delivery of particulate medicaments in that the particles can agglomerate during storage. Agglomeration of the medicament leads to inefficiency in delivery, as a greater proportion of the medicament fails to reach the lungs due to increased particle mass. Agglomerated particles may also lead to difficulties in medicament absorption through the mucous membranes of the respiratory system, and possible irritation.

WO 96/22802 alleviates some of these problems by using a single dose inhaler having a tubular body in which an airflow passage is defined, which extends substantially to the back of the oral cavity, and as far as the trachea inlet. A single dose of an active, inhalable, particulate substance is arranged within the airflow passage and is sealed or closed in relation to the ambient atmosphere by closure means, such as removable caps. The device has a relatively small cross-sectional flow passage area (70 mm²) along the length of the flow passage to promote a high flow velocity during inhalation.

Whilst reducing the potential for the pharmaceutical agent to be deposited in the oral cavity of the user, the inhaler of WO 96/22802 extends to the back of the throat of the user, and can be uncomfortable during use. Furthermore, the device must be manipulated such that it appropriately delivers the medicament to the back of the throat. This can result in incorrect administration and thus the medicament is largely deposited in the oral cavity as opposed to the user's lungs. WO 96/22802 fails to address the problems of particle agglomeration.

EP 0525720 describes an inhalation device for oral and nasal inhalation of medicaments that can repeatedly permit the inhalation of medicament through an inhalation passageway. The passageway may comprise a venturi to increase air velocity within the passageway. The inhalation device is adapted to receive a foil-sealed dosage capsule of powdered medicament. A piercing needle having a bore therethrough extends into the inhalation passageway, and is adapted to pierce the body of the dosage capsule when in use. The user must then manually remove the foil-seal of the dosage capsule, to create a second orifice in the capsule, thereby enabling air to pass through the capsule between the previously sealed opening, and the orifice made by the piercing needle. A sharp inhalation causes air to flow through the air passageway, reducing air pressure in the passageway which causes air to be drawn through the container and the bore of the piercing needle into the air passageway.

However, the inhaler device of EP 0525720 fails to prevent a significant proportion of the medicament being deposited in the oral cavity, and does not address the problem of particle agglomeration. The inhaler device of EP 0525720 generally requires both hands to remove the foil covering of the capsule, and this task can involve an excessive level of dexterity for some users, particularly the elderly. Having to remove the foil covering of the capsule can also lead to medicament loss or contamination.

WO2005/004962 (Cipla Limited) describes a multiple dose inhaler device, in which a piercing portion forms an orifice in a cavity of medicament. The piercing portion forms a seal around the orifice with the pierced material.

It is an object of the present invention to provide a simple, easy to use, compact, lightweight and portable inhalation device for administering a particulate medicament orally or nasally. More particularly, it is an object to provide an inhaler device which can be operated with a single hand.

It is another object of the invention to increase the medicament delivery to lungs by reducing the deposition of drug in the oral cavity and in the inhaler.

It is another object of the invention to increase the deaggregation of agglomerated particles of medicament to achieve the object of increased medicament delivery to lungs.

The present invention solves or alleviates the problems of the prior art.

In broad terms, the inhaler device of the present invention is convenient to use, re-useable, compact, lightweight and portable. It can be used with a single hand, i.e. held between thumb and index or fore finger.

In a first aspect, the invention provides a device for the inhalation of a particulate substance by a user, the device comprising: a housing having first and second openings, a first air passageway being defined through said housing between said first and second openings, and an airflow directing means proximate one of said first and second openings.

The housing may be generally tubular. The first and second openings may be formed at, or proximate to, first and second ends, respectively.

In use, on inhalation by a user, the airflow directing means may act to influence the air pressure differential between within the air passageway and outside the housing.

The airflow directing means may control the amount of air entering the first air passageway. The airflow directing means may control the velocity of air entering the first air passageway. The airflow directing means may restrict the flow of air into the air passageway. Preferably, the airflow directing means restricts airflow into the first air passageway through one of the first or second ends, when in use, on inhalation by a user. The airflow directing means may also be adapted to impart a direction to air flowing into or out of the first air passageway.

Preferably the airflow directing means comprises at least one member to impart turbulence and/or directional flow to air flowing into or out of the first air passageway. In particular, the airflow directing means may impart a helical or whirling motion to air entering the first air passageway.

The airflow directing means may be integrally formed, in the housing, optionally proximate one of said first and second ends of the housing. Alternatively, the airflow directing means may be removably attachable to one of the first or second ends of the body. Preferably the airflow directing means is removably attachable to the second end of the body.

In another embodiment, the airflow directing means may be arranged within the air passageway. The airflow directing means may be arranged in a direction substantially perpendicular to the longitudinal axial direction of the air passageway. The airflow directing means may extend partially or fully across the air passageway.

The airflow directing means may comprise an air channel. The air channel may permit air to pass through the airflow directing means into the air passageway. The air channel may extend in a direction parallel to the axis of air passageway, or may extend in a direction at an angle to the axis of the air passageway.

The airflow directing means may comprise a cap over an end of the housing. The cap may obstruct airflow along the air passageway.

The airflow directing means may comprise a plug in the air passageway. The plug may obstruct airflow along the air passageway.

The airflow directing means preferably comprises at least one air channel. In one embodiment, the airflow directing means comprises one or more members or blades, which may be angled to impart a specific direction to the airflow into the device. The air channels may be formed between said blade(s). The air channels may be helically orientated through the airflow directing means. The blades may be rotationally oriented about a central axis. The blades may be rotationally spaced apart.

In a preferred embodiment, the airflow directing means comprises a cap over an end of the housing, the cap having formed therein a plurality of air channels. The air channels are preferably blades rotationally oriented about a central axis. The blades may extend across the air passageway, such that air passing through the air passageway must therefore pass through channels formed between the blades.

The resistance, turbulence and/or direction of the flow of air entering the passageway will depend on the number of blades/air channels in the airflow directing device.

The number of blades/air channels may vary between 2 and 12 depending on the characteristics of the medicament powder, for example bulk density, cohesiveness, flow of the blend, etc. In one embodiment, the airflow directing means comprises four air channels separated by four angled blades. In another embodiment, the airflow directing means comprises six air channels separated by six angled blades.

In another embodiment, the airflow directing means comprises a mesh. The mesh may overlie an end of the device, or may be arranged within the air passageway. Typically the mesh comprises an array of perforations. The perforations of the mesh may extend at an angle to the longitudinal axis of the air passage, or may extend in a direction parallel to the longitudinal axis of the air passage.

In another embodiment, the airflow directing means may comprise an obstruction to the air passageway. The obstruction may comprise a cover positioned in the air passageway or arranged at an end of the air passageway. The obstruction may influence the airflow into and/or out of the air passageway. The obstruction may comprise a disk. In an embodiment, the obstruction may have a plurality of slits formed therein which permit air to enter or pass along the air passageway. In an embodiment, the obstruction comprises a louvre. The louvre comprises at least one or a plurality of slats. The slats may define longitudinal airflow passages therebetween which permit airflow between into or out of the air passageway. The slats may be angled with respect to a longitudinal axis of the air passageway.

In another embodiment, the airflow directing device comprises one or more helical grooves.

In one embodiment, an inner surface of the first air passageway is in the form of a venturi. The constriction of the venturi increases air velocity within the first air passageway, resulting in a drop in pressure. This effect is further influenced by the configuration of the airflow directing means. As described above, in use, the airflow directing means influences the pressure drop on inhalation between the ambient air pressure and the air pressure within the first air passageway. The use of a venturi together with the airflow directing means, in use, further influences the pressure differential between the air pressure within the air passageway and the ambient air pressure.

Preferably, in the abovedescribed embodiment, the device further comprises an extraction member having an opening formed therein and a second air passageway extending through said extraction member in said opening. The second air passageway may converge with the first air passageway at a convergence point. Preferably, the convergence point is proximate constriction of the venturi, so as to enhance the pressure differential between the air at the convergence point within the first air passageway and the air proximate an end of the extraction member.

The use of an airflow directing means together with the venturi reduces the air pressure at the convergence point, thereby influencing the flow of air through the extraction member. The use of an airflow directing means together with the venturi may enhance the flow of air through the extraction member.

The length, diameter and constriction of the venturi will depend on the characteristics of the particulate material, the desired dosage, etc. In a preferred embodiment, the diameter of the venturi is from 2 mm to 10 mm. Typically, the length of the venturi is between 5 mm and 25 mm. In a preferred embodiment, the length of the venturi is approximately 11 mm.

In one embodiment, the air passageway is defined by an inner wall of the housing, which is provided with turbulence generating means. In an embodiment, the turbulence generating means may comprise a means to deaggregate the particulate substance.

In one embodiment, the turbulence generating means comprises at least one groove in the inner wall. In a preferred embodiment, the turbulence generating means comprise at least one helical grove. The turbulence generating means disrupts the flow of air through the flow passageway, thereby deaggregating the particles of said particulate substance. The helical groove(s) preferably extends from a position in the first air passageway proximate the convergence of the second air passageway with the first air passageway, and extends to proximate the end of the body, which, in use, is inserted into the mouth or nose of a user. The helical groove(s) may be separated from an adjacent turn of the (or another) groove by a ridge. The or each ridge may have a first angled face projecting perpendicularly to the longitudinal axis of the air passageway, or projecting back towards the end of the device which, in use, is open to the atmosphere. The or each ridge may further comprise a second angled face projecting towards the end of the device which is adapted to be inserted into the mouth or nose of a user. Where the turbulence generating means comprise at least one helical grove, suitably the size (amplitude) of the grooves may vary from 0.1 mm to 5 mm. Preferably the size of the grooves is 1 mm.

In one embodiment, the diameter of the air passageway is approximately 5 nm (between the ridges), or approximately 7 mm (between the grooves).

In a preferred embodiment, the turbulence generating means extends on the inner wall of the air passageway between the constriction of the venturi and the end of the body which is, in use, inserted into the nose or mouth of a user.

The turbulence generating means may comprise a mesh or a perforated member, positioned within the air passageway. The mesh or perforated member may be used alone or in combination, and may be combined with the abovedescribed groove.

In another aspect, the present invention provides a device for the inhalation of a particulate substance by a user, the device comprising: a housing comprising an extraction member having an elongate body with a first air passageway formed therethrough; wherein said extraction member is adapted, in use, to penetrate a cartridge containing said particulate substance such that airflow into said cartridge is permitted about a periphery of said extraction member whilst the cartridge remains penetrated by the extraction member.

In an embodiment, the extraction member has a bulbous end and has an opening formed therein; said air passageway extending through said extraction member to said opening.

Typically, the particulate substance will be a medicament, and will be held within a cartridge. Typically the cartridge will be a plastic cartridge, although the cartridge may be made of any suitable material. The cartridge will typically contain a single dose of said particulate substance. Typically, the cartridge will be sealed during manufacture with a covering. Typically, the covering will be piercable by the extraction member. Typically this covering will be foil or plastic, although the invention is not limited to any particular covering.

The device may be supplied with said cartridge, which may be in cooperation with said device, or separate therefrom. In an embodiment, the device is supplied to the user with the cartridge in a pre-actuation position.

In an alternative embodiment, the particulate matter may be held in a blister or a battery of cartridges, or a capsule. The capsules could be replaceable, and could be positioned within the cartridge when required. The pierced capsule could be discarded once used.

Preferably, the shape of the extraction member enables it to penetrate a cartridge containing the particulate substance, thereby forming a single orifice in the cartridge. Typically the orifice will be formed in the covering/seal of the cartridge. The shape of the extraction member preferably enables airflow into the cartridge through the orifice about a periphery of the extraction member, when there exists a low pressure (i.e., lower than ambient air pressure) in the air passageway. Preferably the shape of the piercing end of the extraction member will promote air entering the cartridge to circulate about the cartridge. The air entering the cartridge through said orifice will intermix with the particulate substance in the cartridge, and will be drawn through the opening in the extraction member and into the air passageway in the extraction member.

In one embodiment, the extraction member has an elongate tubular body having grooves or channels in an outer surface thereof. The grooves/channels enable airflow into the cartridge during use, through the orifice formed in the capsule.

In a preferred embodiment, the extraction member has an elongate body and a tip with a greater diameter than that of the elongate body. More preferably, the extraction member has an elongate body and a bulbous end. The bulbous end creates an enlarged orifice in the cartridge during use, but is located within the cartridge after actuation. Thus, air is permitted to enter the cartridge freely about the periphery of the elongate body. The bulbous end also promotes air circulation within the cartridge during inhalation, and thus enhances entrainment of the particulate substance.

The elongate body of the extraction member suitably has an air passage diameter of 1 mm to 3 mm. Preferably the elongate body of the extraction member has a diameter of approximately 1.5 mm. Typically, the extraction member is between 2 mm and 10 mm in length. Preferably, the extraction member is approximately 4 mm long. The characteristics of the extraction member (length, width, diameter, diameter of air passageway, etc. may be varied to suit the characteristics of the particulate substance, the fill weight of the particulate substance in the cartridge, the desired dosage, etc. The extraction member may extend in a direction substantially perpendicular to the housing. Preferably, the cartridge is shaped such that it can be slidably received on said housing. The cartridge preferably comprises a cavity for the storage of said particulate material.

Preferably the cavity of the cartridge is sealed prior to use. The seal may be comprised of any suitable material. Preferably, the seal is a layer of aluminium foil. The cartridge preferably has first and second running surfaces which cooperate with the housing to permit reciprocal movement therebetween. The size and dimensions of the cavity within the cartridge are dependent on the particulate substance and the dosage requirements. In one embodiment, the depth of the cavity may vary from 2 mm to 15 mm. However, the depth of the cavity of the cartridge will vary depending on the final blend characteristics of the particulate substance and the fill weight of the particulate substance in the cartridge.

In one embodiment, the inhaler device comprises support means extending from the body. In a preferred embodiment, the support means may cooperate with a cartridge containing said particulate substance. The support means preferably provides a running surface along which the cartridge is slidably moveable. In an embodiment, the cartridge is removable from the support means. In this embodiment, the device may be used repeatedly by replacement of the cartridge in cooperation with the support means.

Preferably the support means extends substantially perpendicular to the housing. More preferably, the support means extends in a direction parallel to the direction at which the extraction member extends from the housing.

In one embodiment, the support means and/or the cartridge comprises at least one stop member, and the other of the cartridge and the support means comprises at least one abutment for cooperation with the at least one stop member. In a preferred embodiment, one of the cartridge and the support means comprises two of said stop members. In this preferred embodiment, the cartridge is moveable between a first, pre-actuation, position in which the extraction member is proximate, but has not pierced, the cartridge, and a second, predetermined actuation, position in which the extraction member has pierced the cartridge. In the predetermined actuation position, the inhaler device is ready for inhalation of the particulate substance by a user.

In the actuation position, the device is typically ready for dosage delivery. In this position, airflow is permitted about the periphery of the extraction member.

When the device is in cooperation with a cartridge, the cartridge may be movable on activation by a user into a predetermined actuation position in which the bulbous end of the extraction member extends into the cartridge. In an embodiment, the device is supplied to the user with the cartridge in the pre-actuation position.

In the predetermined actuation position, in which the bulbous end of the extraction member is located within the cartridge, the elongated body of the extraction member may extend from within the cartridge through the orifice formed in the covering to the exterior of the cartridge.

It will be appreciated that, although various aspects of the invention have been independently described above, all of the features the present invention may be utilised alone or in combination. For example, the extraction member may be utilised in combination with the airflow directing means, the turbulence generating means or the venturi, together or independently of one another.

The inhaler of the present invention may be made, of any suitable material, such as plastic, metal, antistatic plastic, biodegradable materials, etc. In a preferred embodiment, the device is disposable. In this embodiment, the device may be supplied with a single cartridge of particulate substance, in the abovedescribed pre-actuation position.

The present invention also provides a device for the inhalation of a particulate substance by a user, the device comprising a housing having first and second ends, and an air passageway being defined through said body between said first and second ends.

In an embodiment, the device further comprises an extraction member extending from the housing, the extraction member having an elongated body and a bulbous end and having an opening formed therein; and wherein said air passageway extends through said extraction member to said opening.

In another aspect of the invention, there is provided the use of a device according to any preceding claim for the administration of a particulate substance to the respiratory system of a patient.

The invention will be further described with reference to the drawings in which:

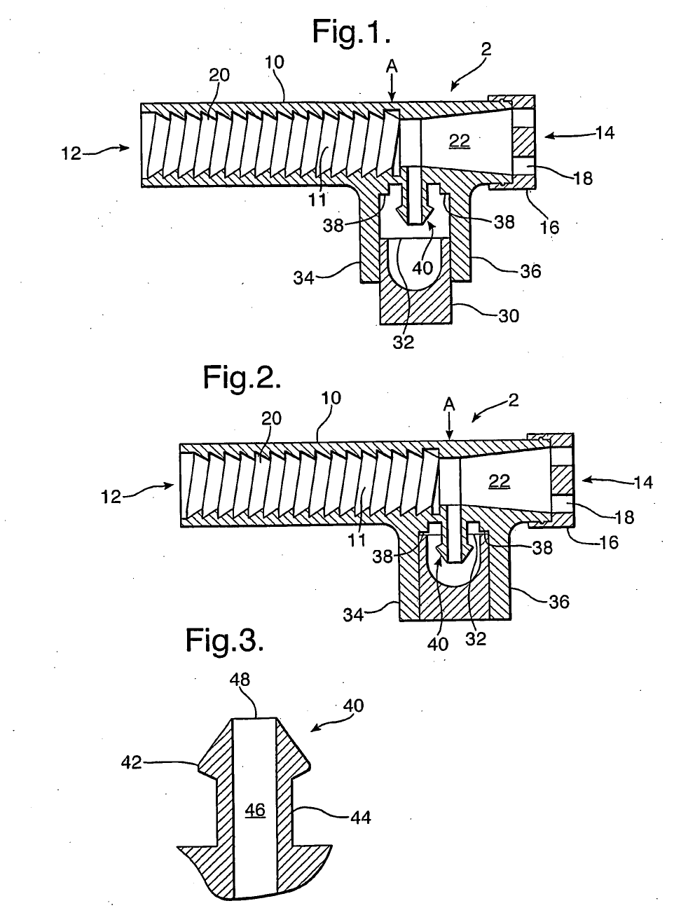

FIG. 1 is a cross-sectional view of an inhaler according to an embodiment of the present invention in a pre-actuation position;

FIG. 2 is a cross-sectional view of the inhaler of FIG. 1 in an actuation position;

FIG. 3 is a cross-sectional view of the extraction member of the inhaler of FIG. 1;

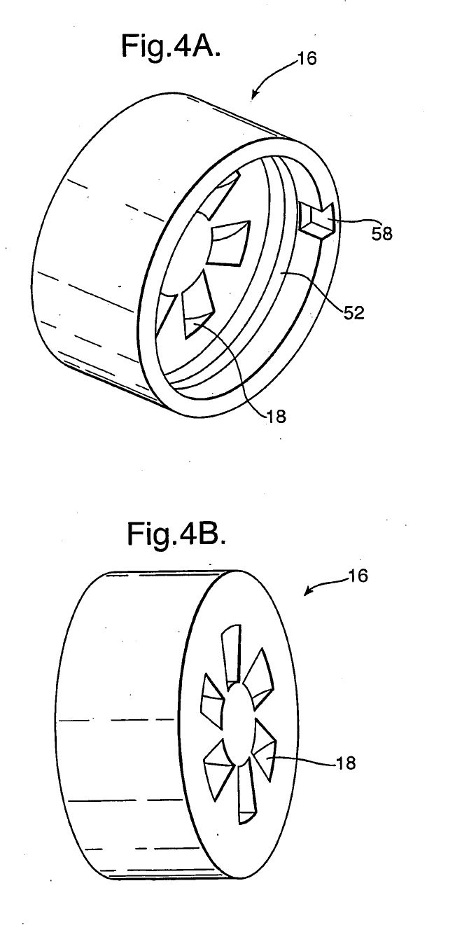

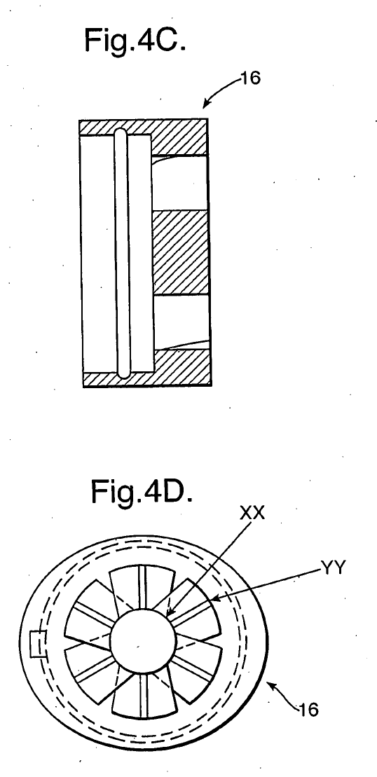

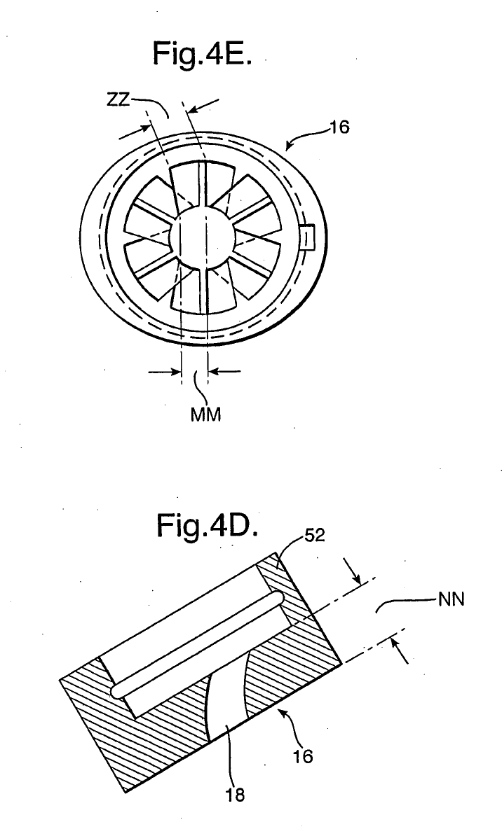

FIGS. 4A to 4F depict various views of an airflow directing means according to an embodiment of the present invention;

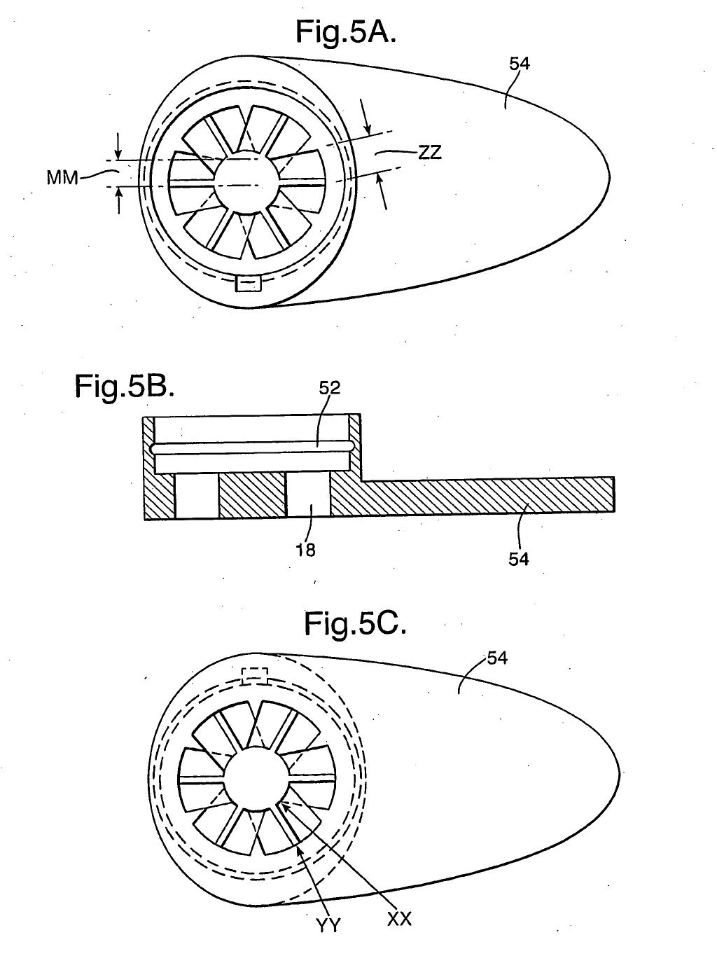

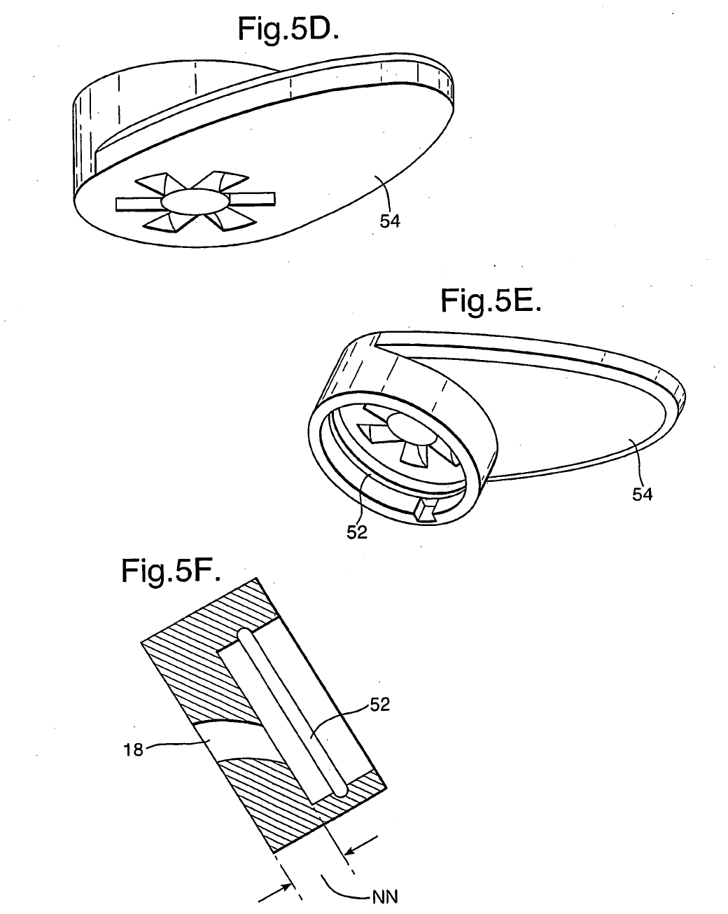

FIGS. 5A to 5F depict various views of an airflow directing means according to an embodiment of the present invention;

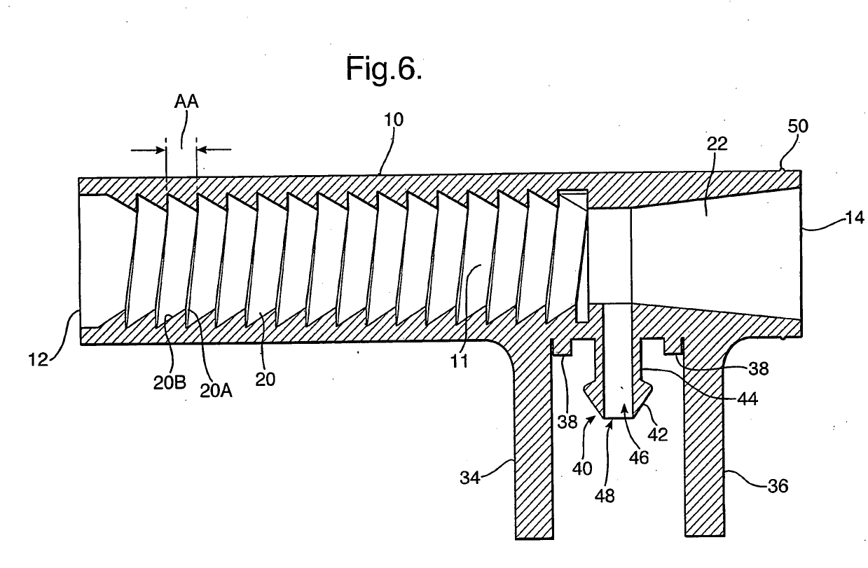

FIG. 6 is a cross-sectional view of an inhaler according to an embodiment of the present invention with the cartridge removed; and

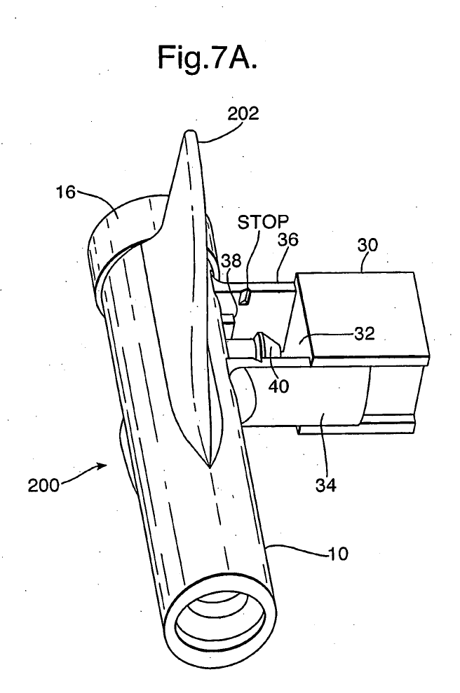

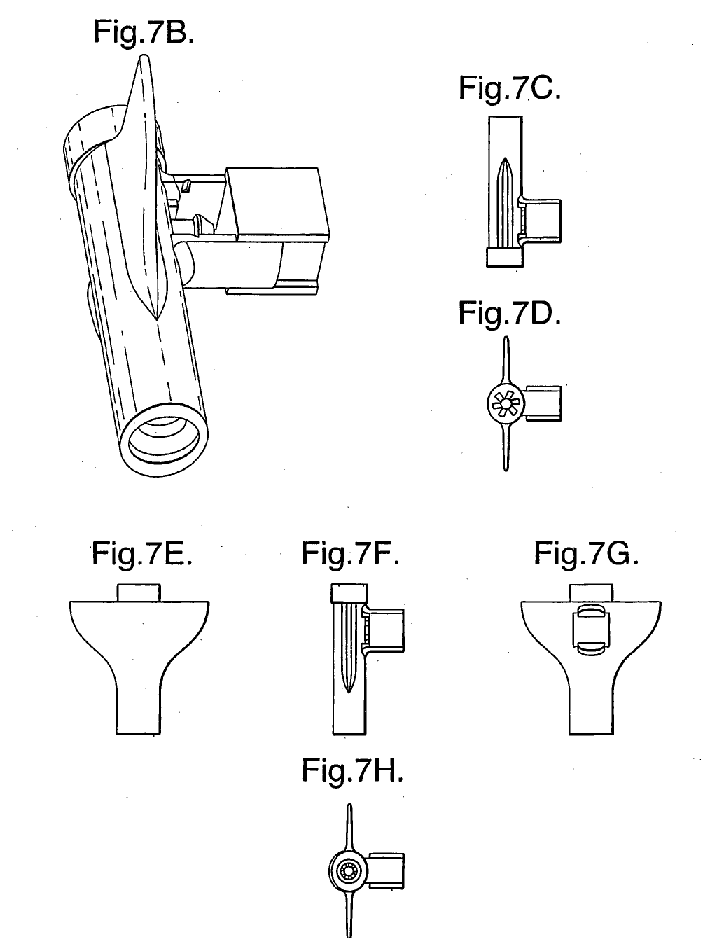

FIGS. 7A to 7H depict various views of a preferred embodiment of an inhaler device according to the present invention.

The present invention provides an inhaler device which is compact and easy to use. The parameters of the inhaler may be varied depending on the substance to be inhaled. For example, the configuration of the airflow directing means, the turbulence generating means, the venturi, the extraction member, the size of the cartridge, the parameters of the cavity within the cartridge, the location of the extraction member and the size of the housing, may all be varied to conform with the substance to be inhaled and the dosage required.

A cross-sectional side view of an inhaler device 2 according to the present invention is shown in FIGS. 1 and 2 of the accompanying drawings. The inhaler device 2 comprises a substantially tubular body 10 defining a first air passageway 11, extending along a longitudinal axis of the body 10. The body has a first end 14, which, in use, is open to the atmosphere, and a second end 12 which is adapted to be inserted into the mouth or nostril of a user.

The first end 14 is provided with a cover or airflow directing means 16, which, in the depicted embodiment, is releasably attachable to the body 10. The cover 16 comprises plurality of angled or helical channels 18. In use, channels 1 split the incoming air stream and causes a swirling motion of air, when air is inhaled through the second end 12 of the tubular body 10. The channels 18 also restrict the volume of air entering the air passageway 11, resulting in a decrease in air pressure within the air passageway 1.

Proximate to the first end 14 is a venturi 22, which, in use, on inhalation by a user, enhances the a pressure drop proximate the point A of said venturi 22 at which the air passageway has the smallest diameter. Extending from the venturi 22 to the second end 12 of the tubular body 10 is a helical grove 20. Groove 20 has a asymmetric profile, having a relatively low inclined face when viewed from the second end 12 of body 10, and a relatively high inclined face when viewed from the first end 14 of body 10. This particular formation enables helical grooves 20 to break the flow of inhaled air. The steeply inclined faces of the groove 20 assists in the deaggregation of the particles of composition being inhaled.

End cap or airflow directing means 16 is provided with angled blades which separate the plurality of air channels 18 thereby splitting the stream of air entering the first end 14. The end cap controls the amount of air entering air passageway 11, on inhalation by user, and hence controls the velocity of air entering the air passageway 11. The turbulence and motion of the air will depend on the number of, and angle of, the blades of the end cap 16. The configuration of the blades is such that it gives a desired pressure drop across venturi 22. The number of blades may vary from two to twelve depending on the desired pressure drop and the blend characteristics of the medicament powder. For example, bulk density, cohesiveness, flow of the blend, etc.

The particulate substance is housed within a cartridge 30. Cartridge 30 is generally in the form of a blister pack containing a single dose of said particulate substance, and sealed with a foil seal 32. Cartridge 30 is slidably moveable along first and second support arms 34, 36. Cartridge is also removable from support arms 34, 36. Formed on the body 10 proximate the point where the support arms 34, 36 meet the body 10, are first and second abutments 38, which halt movement of cartridge 30 when it is plunged towards to body 10. Abutments 38 thereby maintain the cartridge 30 a sufficient distance from the body 10 in the actuated position such that air can flow freely into the cartridge 30 between the foil seal 32 of the cartridge 30 and the body 10. Further stop members (not shown) on the cartridge 30 and the support arms 34, 36, cooperate to provide the cartridge with controlled movement between a first, pre-actuation position, as show in FIG. 1, and a second, actuated position, as shown in FIG. 2.

In the depicted embodiment, the cartridge 30 is a container or capsule of particulate medicament. The container is shaped such that it cooperates with the body 10 and is slidably moveable relative thereto. In another embodiment, the cartridge 30 may be a receptacle for a separate capsule of medicament, which may be replaceable.

An extraction member 40 extends from the body 10 between the support arms 34, 36. The extraction member 40 will be described further with reference now to FIG. 3. Extraction member 40 has an elongate body 44 and a tip 42 having a greater diameter than that of the body 44. A second air passageway 46 extends along the length of the extraction member 40 from opening 48. The second air passageway 46 converges with the first air passageway 11 proximate the narrowest point A of the venturi 22.

Referring again to FIGS. 1 and 2, the operation of the embodiment will be described. Pressure is applied to the cartridge 30, which is in the pre-actuation position depicted in FIG. 1 (in which a first stop member (not shown) on the support arms 34, 36 interacts with an abutment (not shown) on the cartridge 30). In this position, there is a gap between the extraction member and the foil seal 32 of the cartridge, and the cartridge 30 is intact. When required, the user plunges the cartridge (generally between finger and thumb) from its pre-actuation position to its actuation position, which is depicted in FIG. 2.

In the actuation position, the cartridge 30 has been depressed (in this position, an abutment (not shown) on the cartridge 30 interacts with a second stop member (not shown) on the support arms 34, 36, and an end of the cartridge 30 interacts with the abutments 38 on the body 10). In this position, the extraction member has pierced the foil seal 32 of the cartridge 30, the tip 42 of the extraction member is located internally in the cartridge 30, and the elongate body 44 of the extraction member extends through an orifice made in the foil seal 32. The device is now ready for inhalation.

On inhalation by the user through the second end 12, the pressure decreases within the air passageway 11 to below atmospheric pressure. Air is drawn into the first air passageway 11 through channels 18, which impart a swirling movement to the air in venturi 22. The size and shape of the channels 18 act to restrict the influx of air into the air passageway 11. Due to the shape of the venturi 22, air velocity increases to the point where the first and second air passageways 11, 46 converge (i.e. at the constriction A of the venturi 22). This reduces the air pressure proximate this point further, enhancing the pressure differential between the air at the constriction A and the ambient air pressure. Air flow is thus enhanced through air passageway 46, via the cartridge 30. Air enters the cartridge 30 through the pierced foil seal 32 about the periphery of the elongate body 44, and is directed outwardly about the cartridge by the shape of the tip 42. Thus air circulates inside the cartridge 30 entraining the particulate substance therein.

Air entrained with said particulate substance enters the second air passageway 46 and reaches the first air passageway 11 proximate point A. The flow of particulate material continues along the passageway 11, and is broken by contact with helical groove 20, thereby de-aggregating the particles.

With reference now to FIGS. 4A to 4F, various views of a preferred airflow directing means 16 are shown. FIGS. 4A and 4F are isometric views of the airflow directing device, which is adapted to be connected to an end of the housing 10, by means of a snap-fit between groove 52 (FIGS. 4B and 4E) with a raised ring (50 in FIG. 6) on the housing 10. With reference to FIG. 4A, orienting profile 58 on the airflow directing means 16 interacts with a correspondingly shaped profile on the body 10 to ensure correct alignment of the airflow directing means 16.

The helical orientation of the air channels 18 is clearly shown in FIG. 4C.

With reference to FIGS. 4D-4F, the parameters of a preferred embodiment of the airflow directing device 16 is shown. The air channels 18 in FIG. 4D extend from the point XX at a diameter of 3.0 mm to point YY at a diameter of 7.0 mm. In FIG. 4E the six air channels have an exterior width at point ZZ of 1.6 mm, and a interior width at the point MM of 1.2 mm. With reference to FIG. 4F, depth of the end cap 16 at point NN is 2.0 mm.

FIGS. 5A to 5F show various views of another preferred airflow directing means comprising 6 channels separated by angled blades. The airflow directing means has an elongate lip 54 to prevent swallowing and/or inhalation. The parameters of the channels 18 of this device are identical to that described in FIGS. 4D-4F.

FIG. 6 shows a cross-section of a preferred embodiment of an inhaler device of the present invention, in the absence of the cartridge 30 and airflow directing means 16. The profile of the helical groove 20 is clearly shown in FIG. 6. In the depicted embodiment, the angle of the first angled face 20A is approximately 90 degrees to the longitudinal axis of the air passageway 11, and the angle of the second face 20B is approximately 34 degrees to the longitudinal axis of the air passageway 11.

The pitch of the helical groove 20, represented by AA is approximately 1.6 mm. The maximum diameter of the air passageway 11, from trough to trough of groove 20, represented by BB, is approximately 7.0 mm, and is approximately 5.0 mm, from peak to peak, represented by CC.

In the device as shown, the length of the venturi is 11 mm. The length between the extraction member/constriction (i.e. point A) and the second end 12 is typically between 5 mm and 55 mm, although preferably the length is 28.5 mm+/−5 mm. However, it will be clearly understood that the dimensional parameters of the device will be variable.

FIG. 7A is an isometric view of a preferred embodiment of an inhaler device 200 according to the present invention in its pre-actuation position. The inhaler device 200 comprises wings 202 extending from the body 10, which prevent or minimise the risk of inhalation or swallowing. In its pre-actuation position, an abutment on cartridge 30 interacts with a stop member (not shown) on the support arms 34, 36 to maintain the cartridge 30 in this position. In use, the user would depress the cartridge 30 by applying sufficient force to, the cartridge 30 to move the abutment out of correspondence with the stop member (not shown), such that the cartridge moves to its actuation position in which the extraction member 40 has pierced the foil seal 32 of the cartridge 30. The cartridge is held in the actuation position by stops 38 on the housing 10, and by the stop member STOP formed in the support arms 34,36.

FIGS. 7B to 7H are schematic views of the device in its actuation position. Inhalation in this position, as described above, creates an air influx through air channels 18 and air passageway 46 into air passageway 11. The configuration of the air channels 18/blades is such that it results in a desired pressure drop across the venturi 22, and thus enhances entrainment of the medicament present within the capsule 30. The medicament is thus drawn into the air passageway 11, and towards the mouth/nostril of the user. Turbulence created by the groove and contact between the medicament and the groove 20 (particularly face 20A) reduces agglomeration of the medicament particles, further enhancing the inhalation of the medicament.

It will be appreciated that the invention described above can be modified within the scope of the appended claims.

1-61. (canceled)

62. A device for the inhalation of a particulate substance by a user, the device comprising: a housing having first and second openings, a first air passageway being defined through said housing between said first and second openings, an airflow directing means proximate one of said first and second openings, an extraction member extending from said housing and having a third opening formed proximate an end thereof, and a second air passageway extending through said extraction member and being in fluid communication with the first air passageway.

63. A device according to claim 62, wherein said airflow directing means serves to define the direction of airflow from the exterior of the housing into the first air passageway, wherein said airflow directing means comprises a plurality of air flow channels which extend in a direction at an angle to the axis of the first air passageway, whereby air drawn into the first air passageway through said airflow directing means is directed by said airflow directing means in a direction which is at an angle to said first air passageway.

64. A device according to claim 62, wherein said extraction member is adapted, in use, to penetrate a cartridge containing said particulate substance such that airflow into said cartridge is permitted about a periphery of said extraction member while the cartridge remains penetrated by the extraction member.

65. A device according to claim 64, wherein, in use, on inhalation by a user, air is permitted to flow into said cartridge about a periphery of said extraction member, and is permitted to flow into said first air passageway via said first opening.

66. A device according to claim 62, wherein said second air passageway converges with said first air passageway at a convergence point.

67. A device according to claim 66, wherein the convergence point of the first and second air passageways is proximate a position in the first air passageway at which the first air passageway is at its narrowest diameter.

68. A device according to claim 67, wherein said turbulence-generating means extends substantially from said convergence point to said first opening.

69. A device according to claim 62, wherein said end of the housing opposite the airflow directing means is adapted for insertion into a mouth or nostril of a user.

70. A device according to claim 62, wherein the airflow directing means is integrally formed in, or proximate to, the second opening.

71. A device according to claim 62, wherein the airflow directing means is removably attachable to the housing.

72. A device according to claim 62, wherein the airflow directing means overlies the second opening.

73. A device according to claim 72, wherein each of said air channels is separated from an adjacent air channel by a blade.

74. A device according to claim 73, wherein the blades are angled.

75. A device according to claim 74, which comprises from two to twelve blades.

76. A device according to claim 75, which comprises four blades.

77. A device according to claim 75, which comprises six blades.

78. A device according to claim 62, wherein the airflow directing means comprises a mesh covering.

79. A device according to claim 62, wherein, in use, the airflow directing means restricts the flow of air entering the first air passageway.

80. A device according to claim 62, wherein the first air passageway is defined by an inner surface of said housing, and wherein at least one turbulence-generating means is formed in said inner surface.

81. A device according to claim 80, wherein said turbulence-generating means is proximate said first opening.

82. A device according to claim 81, wherein said turbulence-generating means comprises at least one groove.

83. A device according to claim 82, wherein said turbulence-generating means comprises at least one helical channel formed in the inner surface of said first air passageway.

84. A device according to claim 83, wherein in use, on inhalation by a user, a rotational direction of airflow in said first passageway imparted by the airflow directing means corresponds to a rotational direction of the helical channel.

85. A device according to claim 84, wherein, in use, on inhalation by a user, the turbulence-generating means deaggregates said particulate substance.

86. A device according to claim 62, wherein the first air passageway is defined by an inner surface of said housing, and wherein at least one venturi is formed in the inner surface of first air passageway.

87. A device according to claim 86, wherein said venturi is proximate the second opening.

88. A device according to claim 86, wherein an internal diameter of the venturi is from 2 mm to 10 mm.

89. A device according to claim 88, wherein said venturi extends substantially from the second opening to said convergence point.

90. A device according to claim 62, further comprising a cartridge containing said particulate substance.

91. A device according to claim 90, wherein said cartridge is slidably received on said housing.

92. A device according to claim 62, further comprising support means extending from said housing.

93. A device according to claim 92, wherein said support means extends in a direction substantially parallel to said extraction member.

94. A device according to claim 92, wherein said support means are adapted to receive a cartridge of particulate substance.

95. A device according to claim 94, wherein said support means comprises first and second arms extending from said housing.

96. A device according to claim 95, wherein, in use, a cartridge of particulate substance is slidably received in said support means.

97. A device according to claim 96, wherein, in use, said cartridge is moveable on activation by a user into a predetermined actuation position in which the first opening is positioned within said cartridge.

98. A device according to claim 97, wherein, in use, in said predetermined actuation position, the elongate body of the extraction member extends from within said cartridge to an exterior of said cartridge.

99. A device according to claim 98, wherein said support means comprises at least one stop member for cooperation with at least one abutment on a cartridge of particulate substance.

100. A device according to claim 98, wherein said support means comprises at least one abutment for cooperation with at least one stop member on a cartridge of particulate substance.

101. A device according to claim 100, which comprises first and second stop members.

102. A device according to claim 101, wherein, in use, the cartridge is slidably moveable between a pre-actuation position, in which said abutment cooperates with said first stop member, and said predetermined actuation position, in which the abutment cooperates with said second stop member.

103. A device according to claim 102, wherein, in use, the first opening is spaced apart from said cartridge in the pre-actuation position.

104. A device according to claim 62, wherein the housing further comprises a stop member proximate said extraction member.

105. A device according to claim 62, wherein the extraction member has a tip, said tip having a greater diameter than the diameter of the elongate body.

106. A device according to claim 62, wherein the extraction member has a bulbous end.

107. A device according to claim 62, wherein the extraction member extends from the housing.

108. A device according to claim 62, wherein the first air passageway has a diameter of from 1 mm to 3 mm.

109. A device according to claim 108, wherein the first air passageway has a diameter of 1.5 mm.

110. A device according to claim 62, wherein the extraction member has a length of between 2 mm and 10 mm.

111. A device according to claim 110, wherein the extraction member has a length of 4 mm.

112. A device according to claim 62, wherein the extraction member extends from the housing intermediate said second and third openings.

113. A device according claim 62, wherein the housing has an elongate shape.
